(19) United States
(12) Patent Application Publication
(10) Pub. No.: US 2023/0113716 A1
(43) Pub. Date: Apr. 13, 2023

(54) ALLOY FOR INHIBITING ACTIVITY OF BACTERIAL COLLAGENASE AND/OR MATRIX METALLOPROTEINASE

(71) Applicant: Bio DG, Inc., Poway, CA (US)

(72) Inventors: Anthony J. SENAGORE, Poway, CA (US); William MONTGOMERY, Poway, CA (US); Warren PELISSIER, Poway, CA (US); Gordon F. JANKO, Poway, CA (US)

(21) Appl. No.: 17/961,443

(22) Filed: Oct. 6, 2022

Related U.S. Application Data
(60) Provisional application No. 63/253,925, filed on Oct. 8, 2021.

(51) Int. Cl. C22C 38/04; A61K 9/00; A61L 27/04

(52) U.S. Cl. CPC C22C 38/04; A61K 9/0024; A61L 27/042

(57) ABSTRACT

The present disclosure is directed to a medical device for inhibiting the activity of bacterial collagenase and/or a matrix metalloproteinase (MMP). In some embodiments, the medical device comprises a polymer that includes a transition metal or a salt thereof. In some embodiments, the medical device comprises an alloy that has at least one of a transition metal or a transition metal salt.

### CROSS-REFERENCE TO RELATED APPLICATIONS

The present invention claims priority to and benefit of U.S. Provisional Application No. 63/253,925, filed Oct. 8, 2021, and entitled “Alloy for Inhibiting Activity of Bacterial Collagenase and/or Matrix Metalloproteinase,” the entire disclosure of which is incorporated herein by reference.

### TECHNICAL FIELD

The present invention relates to alloys, medical devices related to surgery, and in particular, medical devices used for tissue fixation/approximation to support surgical and traumatic wound healing.

### BACKGROUND

Healing of surgically or traumatically injured soft tissue and bone requires effective reconstitution of the extracellular matrix (ECM) that includes a combination of proteins and proteoglycans integrated into a dense network important for tissue stability. Collagen is the primary structural protein of the ECM and synthesis of collagen requires a complex process including significant post-translational modifications before a mature collagen fibril is formed. During the healing process, there is a balance between collagen synthesis and collagen degradation as the hydroxylation and glycosylation reactions proceed, followed ultimately by collagen cross-linking to restore normal tissue strength. The molecules which control this healing process are the matrix metalloproteinases (MMPs) and the counter-control molecules called tissue inhibitor metalloproteinases (TIMPs).

The MMPs are a family of at least 24 zinc containing peptidase enzymes, such as collagenases, that are involved with many tissue degradation processes. MMPs are capable of degrading most components of extracellular matrices. MMP's are secreted into the extracellular fluid space after synthesis and stored as inactive proenzymes (pro-MMP), although some may also be resident within cells, such as MMP-8 and MMP-9 which are contained in neutrophils. Conversely, the activity of MMPs is inhibited by the tissue inhibitors of metalloproteinases (TIMPs). MMPs are essential for normal soft tissue healing because selective collagen degradation provides for more orderly collagen remodeling to restore normal tissue strength driven supported by the inhibitory regulation from TIMPs.

While the multiple potential interaction of the myriad MMP's and TIMP's has yet to be fully assessed, there is a growing body of information regarding the negative impact of certain MMP's on wound healing, as well as the up-regulation of these harmful MMP's by bacterial collagenase. This up-regulation serves to further enhance the potential for adverse tissue healing in concert with the harmful effects of bacterial collagenase produced by certain bacteria because of wound contamination and/or infection. The result is that recent data has suggested an association between the combined effect of bacterial collagenase and the resulting up-regulated human MMP's on 3 very compelling adverse clinical outcomes: 1) colorectal anastomotic leak; 2) ventral hernia formation; and 3) fracture non-union. Okamoto, et al. demonstrated that Pro-MMP 1, Pro-MMP 8 and Pro-MMP 9 can be activated by several bacterial proteinases via differing mechanisms, however cleavage of the pro-peptide domain by a bacterial collagenase is a clinically relevant mechanism. These classical collagenases (MMP-1, MMP-8, and MMP-13) degrade type I and III collagen in their native triple-helical form by unwinding them into gelatin. Thereafter, the gelatinases (MMP-2 and MMP-9) cleave the denatured collagens.

Therefore, local influence on the activity of bacterial collagenase, as well as the control of the important MMP's would be clinically important to surgical patients to reduce morbidity and mortality related to adverse wound healing. For example, between 5-15 percent of colorectal resections are complicated by anastomotic leak, often resulting in significant mortality due to peritonitis and sepsis. Ventral hernia formation occurs in at least 10% of midline laparotomies, with increased risk associated with various patient factors including surgical site infection. As mentioned previously, non-union of fractures is also related to the wound contamination/infection which requires long periods of morbidity and the need for complicated readmissions. Although the negative impact of both bacterial collagenase and MMP's has been demonstrated, there are no clinically available options, either pharmacologic or device related, that can circumvent the activity of these molecules at the wound level.

Shogan et al, demonstrated that the commensal bacterium Enterococcus faecalis contributed to anastomotic leaks through the activation of tissue Matrix Metalloproteinase 9 (MMP 9) in intestinal tissues. The same team then demonstrated the ability to mitigate the effects of E. faecalis with an oral supplementation of a phosphate carrier. However, the safety and efficacy of this type of pharmaceutical solution will require much further study to confirm the ability to consistently treat a distal bowel anastomosis without adverse effects. The ability to use enteral delivery to provide a highly localized and topical site of action at the anastomosis, obviously limits application of this approach to non-GI surgical procedures such as ventral hernia or fracture care. Potential direct antimicrobial agents risk unknown impacts on the microbiome as well as the risk of the emergence of multi-drug resistant bacteria and are therefore unlikely to be useful in the background of an effective antimicrobial stewardship program.

### SUMMARY

One aspect of the present disclosure relates to a medical device comprising a polymer that includes at least one of a transition metal or a transition metal salt, wherein under normal physiological conditions, the polymer is configured to release an effective amount of an ion or the salt of the transition metal to a surrounding environment for inhibiting activity of a bacterial collagenase and/or a MMP.

In some embodiments, the medical device comprises a medical device body, and a inhibition layer disposed on the medical device body, the inhibition layer comprising the polymer.

In some embodiments, the at least one of the transition metal or the transition metal salt is embedded homogeneously in the polymer.

In some embodiments, the transition metal is iron (Fe), manganese (Mn), silver (Ag), copper (Cu), or nickel (Ni).

In some embodiments, the transition metal salt includes a divalent cation and is selected from FeCl₂, FeS, FeSe, MnCl₂, MnS, MnSe, CuCl₂, CuS, CuSe, NiCl₂, NiS, and NiSe.

In some embodiments, the transition metal is not cobalt (Co). In some embodiments, the transition metal salt is not CoCl₂.

In some embodiments, the MMP is MMP1, MMP8, or MMP9.

In some embodiments, the medical device is in the form of a bone screw, a bone anchor, a tissue staple, a craniomaxillofacial reconstruction plate, a surgical alloy mesh, a composite surgical mesh consisting of woven alloy and natural or synthetic fibers, a surgical closure, a fastener, a reconstructive dental device, a component of a tissue sealant/glue, or a stent.

In some embodiments, the medical device is implantable.

Another aspect of the present disclosure relates to a method of inhibiting activity of a bacterial collagenase and/or a MMP in a subject in need thereof, the method comprising applying to or implanting in the subject, a medical device of comprising a polymer, as described herein.

Yet another aspect of the present disclosure relates to a method of inhibiting activity of a bacterial collagenase and/or a MMP in a subject in need thereof, the method comprising applying to or implanting in the subject, a medical device that has an alloy, wherein: the alloy has a transition metal or a transition metal salt; and under normal physiological conditions, the alloy is configured to release an effective amount of an ion or the salt of the transition metal to a surrounding environment for inhibiting activity of the bacterial collagenase and/or the MMP.

In some embodiments, the at least one of the transition metal or a transition metal salt is embedded homogeneously in the alloy.

In some embodiments, the transition metal is iron (Fe), manganese (Mn), silver (Ag), copper (Cu), or nickel (Ni).

In some embodiments, the salt of the transition metal includes a divalent cation and is selected from FeCl₂, FeS, FeSe, MnCl₂, MnS, MnSe, CuCl₂, CuS, CuSe, NiCl₂, NiS, and NiSe.

In some embodiments, the transition metal is not cobalt (Co). In some embodiments, the salt of the transition metal is not CoCl₂.

In some embodiments, the MMP is MMP1, MMP8, or MMP9.

In some embodiments, the medical device is in the form of a bone screw, a bone anchor, a tissue staple, a craniomaxillofacial reconstruction plate, a surgical alloy mesh, a composite surgical mesh consisting of woven alloy and natural or synthetic fibers, a surgical closure, a fastener, a reconstructive dental device, a component of a tissue sealant/glue, a suture, and/or a stent.

In some embodiments, the alloy comprises at least 50% iron by weight, at least 25% manganese by weight, and at least one of 0.01% sulfur or selenium by weight, and wherein the alloy is nonmagnetic.

In some embodiments, the alloy comprises at least 60% Fe by weight.

In some embodiments, the alloy comprises at least 30% Mn by weight.

In some embodiments, the alloy comprises Mn in a range of 28% to 35% by weight, Fe in a range of 65% to 72% by weight, and Nb in a range of 0.1% to 0.3% by weight.

In some embodiments of each aspect described above, the medical device is implanted in the gastrointestinal tract, soft tissue, or bone of the subject.

In some embodiments of each aspect described above, the effective amount of the ion or the salt of the transition metal is about 10 micromolar to about 120 micromolar.

In some embodiments, a medical device comprises a medical device body formed from a transition metal alloy including Mn in a range of 28% to 35% by weight, Fe in a range of 65% to 72% by weight, and Nb in a range of 0.1% to 0.3% by weight, the medical device configured to be implanted in the body of a patient or applied to a tissue of the patient such that under normal physiological conditions, the medical device body is configured to release an effective amount of the Mn and Fe in the surrounding tissue for inhibiting activity of a bacterial collagenase and/or a matrix metalloproteinase (MMP).

In some embodiments, the effective amount of Fe and Mn in the surrounding tissue is in a range of about 10 μM to about 120 μM. In some embodiments, the effective amount of Fe and Mn in the surrounding tissue is in a range of about 40 μM to about 80 μM after at least 3 days of the medical device being in contact with the surrounding tissue.

In some embodiments, the medical device is in the form of a bone screw, a bone anchor, a tissue staple, a craniomaxillofacial reconstruction plate, a surgical alloy mesh, a composite surgical mesh consisting of woven alloy and natural or synthetic fibers, a surgical closure, a fastener, a reconstructive dental device, a component of a tissue sealant/glue, or a stent.

In some embodiments, the medical device has a yield strength in a range of about 50,000 psi to about 90,000 psi. In some embodiment, the ultimate yield strength of the medical device is in a range of about 80,000 psi to about 150,000 psi.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several implementations in accordance with the disclosure and are therefore, not to be considered limiting of its scope, the disclosure will be described with additional specificity and detail through use of the accompanying drawings.

FIG. 1 is a schematic illustration of a medical device configured to inhibit bacterial collagenase and/or MMP, according to an embodiment.

FIG. 2 is a schematic flow diagram of a method for fabricating a medical device that inhibits bacterial collagenase and/or MMP, according to an embodiment.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

The present disclosure is based, inter alia, on the discovery that the presence of transition metal ions or salts thereof produced as a result of early surface corrosion of an implanted medical device can inhibit the activity of certain enzymes, in particular a bacterial collagenase and/or a matrix metalloproteinase (MMP). Accordingly, to reduce morbidity and mortality related to adverse wound healing in a surgical patient, a medical device implanted in or applied to a skin surface of the patient can include a polymer or an alloy configured to release an effective amount of an ion or a salt of a transition metal to a surrounding environment for inhibiting the activity of a bacterial collagenase and/or a MMP under normal physiological conditions. In some embodiments, the alloy can be embedded in or interwoven with a polymer. Particularly, embodiments of the medical devices including transition metals and/or transition metal salts described herein are configured to release transition metals such as Mn, Fe, etc., in the surrounding tissue. The transition metals replace the Zn that is present in many of the MMPs or proteinases that cause collagen activation, thereby inhibiting the activation of these proteins and thus, inhibiting collagenase activation. The medical devices described herein can continue to release the transition metal ions or salts in the surrounding tissue for a significant period of time (e.g., days or months) after being disposed in the patient such that an effective amount of the transition metal ions remain present the significant period of time in the surrounding tissue to continue inhibition of collagenase and/or MMP in the surrounding tissue.

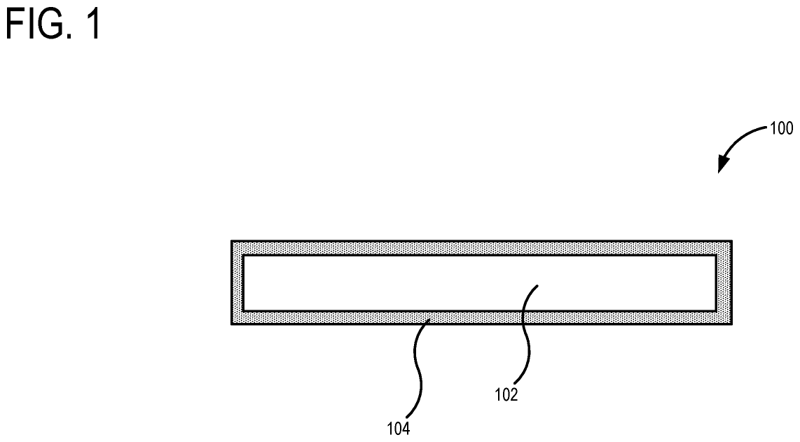

FIG. 1 is a schematic illustration of a medical device 100, for example, an implantable medical device, according to an embodiment. The medical device 100 may be configured to be implanted within the body of a patient or to be applied on the body of the patient, for example, to close a wound on the body of the patient. In some embodiments, the medical device 100 may include a medical device body 102 that may be formed from any suitable material (e.g., metals, alloys, plastics, polymers, ceramics, etc.), and be biodegradable or non-biodegradable. The medical device body 102 may be coated with an inhibition layer 104, that is formulated to inhibit bacterial collagenase and/or bacterial MMP. In some embodiments, the inhibition layer 104 may include a transition metal, an alloy including a transition metal, or a transition metal salt that may, for example, be embedded or incorporated in a polymer. In some embodiments, the transition metal or transition metal alloy may be coated on the medical device body 102 (e.g., via powder deposition, e-beam evaporation, thermal evaporation, etc.). In other embodiments, the transition metal, an alloy including the transition metal, or the transition metal salt may additionally, or alternatively, be embedded or incorporated in the medical device body 102 (e.g., in the form of micro and/or nanoparticles, or alloyed in the medical device body 102 material). In such embodiments, the inhibition layer 104 may be excluded and the medical device body 102 may itself be formulated to inhibit bacterial collagenase and/or bacterial metalloproteinase.

In some embodiments, the medical device body 102 may be formed from the transition metal alloy or transition metal salt. For example, the medical device body 102 may be formed from a transition metal alloy including Mn in a range of 28% to 35% by weight, Fe in a range of 65% to 72% by weight, and Nb in a range of 0.1% to 0.3% by weight. In some embodiments, the transition metal alloy may also include about 0.06% to about 0.1% by weight of carbon. The medical device body 102 may configured to be implanted in the body of a patient or applied to a tissue of the patient such that under normal physiological conditions, the medical device body 102 is configured to release an effective amount of the Mn and Fe in the surrounding tissue for inhibiting activity of a bacterial collagenase and/or a matrix metalloproteinase (MMP). In some embodiments, the medical device is in the form of a bone screw, a bone anchor, a tissue staple, a craniomaxillofacial reconstruction plate, a surgical alloy mesh, a composite surgical mesh consisting of woven alloy and natural or synthetic fibers, a surgical closure, a fastener, a reconstructive dental device, a component of a tissue sealant/glue, or a stent.

In some embodiments, the effective amount of Fe and Mn in the surrounding tissue is in a range of about 10 μM to about 120 μM. In some embodiments, the effective amount of Fe and Mn in the surrounding tissue is in a range of about 40 μM to about 80 μM after at least 3 days of the medical device being in contact with the surrounding tissue. In some embodiments, the medical device has a yield strength in a range of about 50,000 psi to about 90,000 psi. In some embodiment, the ultimate yield strength of the medical device is in a range of about 80,000 psi to about 150,000 psi.

Expanding further, in some embodiments, the medical device 100 or any other medical device described herein, includes a polymer that includes a transition metal(s) or a salt(s) thereof. The polymer is configured to release an effective amount of an ion or the salt (e.g., in a range of about 10 micromolar to about 120 micromolar or ions or salts of the one or more transition metal) of the transition metal to a surrounding environment for inhibiting the activity of a bacterial collagenase and/or a MMP under normal physiological conditions (e.g., at a body temperature of about 37 degrees Celsius). In some embodiment, the polymer including the transition metal or salt thereof may be used to form the inhibition layer 104. In some embodiments, the medical device body 102 may be additionally or alternately, formed from the polymer including the transition metal or salt thereof.

In some embodiments, the transition metal in the polymer can be in the form of an alloy (e.g., alloy particles). In some embodiments, both bioabsorbable and non-bioabsorbable polymer can be used to hold the transition metal or a salt thereof (e.g., an alloy) and provide for early corrosion benefit into the surrounding environment under normal physiological conditions. Biodegradable (also called bioabsorbable) polymers can slowly degrade into the surrounding space, releasing the transition metal or a salt thereof using normal physiologic mechanisms so as to inhibit bacterial collagenase and/or bacterial MMP.

In some embodiments, the polymer includes a biodegradable polymer that may include, but is not limited to, polylactic acid, polyglycolic acid, polylactate-glycolate, polydioxanone, polycaprolactone, a hydrogel, or a combination thereof.

In some embodiments, the polymer includes a non-biodegradable polymer that may include, but not limited to, polyamide, polyethylene, polypropylene, polyurethane, polyester, polytetrafluoroethylene, silicone, polyether block amide copolymer (PEBA), polyethylene terephthalate, polyethylene naphthalate, polyether ether ketone, cotton, silk, or a combination thereof.

In some embodiments, the local inhibition of the activity of a bacterial collagenase, as well as the control of the MMPs due to transition metal ions or salts from the polymer can influence the rate of wound healing at implant or surgical sites. In some embodiments, the MMP can be MMP1, MMP8, or MMP9.

In some embodiments, the polymer is in the form of a coating, for example, forms the inhibition layer 104. In some embodiments, the polymer has a transition metal, a transition metal alloy, or a transition metal salt thereof embedded homogeneously therein. In some embodiments, the coating (e.g., the bacterial inhibition layer 104) on the medical device (e.g., the medical device 100) may be of a predetermined thickness and composition and the composition of the coating may change as a function of the coating thickness. For example, the thickness of the coating can be at least about 50 nm, at least about 100 nm, at least about 150 nm, at least about 200 nm, at least about 250 nm, at least about 300 nm, at least about 350 nm, at least about 400 nm, at least about 450 nm, or at least about 500 nm. In some embodiments, the thickness of the coating can be no more than about 500 μm, no more than about 450 μm, no more than about 400 μm, no more than about 350 μm, no more than about 300 μm, no more than about 250 μm, no more than about 200 μm, no more than about 150 μm, no more than about 100 μm, or no more than about 50 μm. Combinations of the above-referenced ranges for the thickness are also contemplated. For example, in some embodiments, the thickness of the coating is about 50 nm to about 500 μm, about 100 nm to about 400 μm, about 100 nm to about 300 μm, about 100 nm to about 200 μm, about 100 nm to about 100 μm, or about 100 nm to about 50 μm, inclusive. In other embodiments, the medical device may be formed from the polymer. For example, the medical device body 102 of the medical device 100 may be formed from the polymer such that the inhibition layer 104 is excluded. In some embodiments, the medical device body 102 and/or the inhibition layer 104 may be formed from a transition metal alloy, for example, any of the transition metal alloys described herein.

In some embodiments, the transition metal is iron (Fe), manganese (Mn), silver (Ag), copper (Cu), or nickel (Ni). In some embodiments, the transition metal does not include cobalt (Co). In some embodiments, the polymer has a transition metal salt with a divalent cation embedded in the polymer. In some embodiments, the salt of the transition metal is FeCl₂, FeS, FeSe, MnCl₂, MnS, MnSe, CuCl₂, CuS, CuSe, NiCl₂, NiS, NiSe, or AgNO₃. In some embodiments, the salt of the transition metal does not include CoCl₂.

In some embodiments, the medical device including the polymer can be in the form of a bone screw, a bone anchor, a tissue staple, a craniomaxillofacial reconstruction plate, a surgical alloy mesh, a composite surgical mesh consisting of woven alloy and natural or synthetic fibers, a surgical closure, a fastener, a reconstructive dental device, a component of a tissue sealant/glue, or a stent.

In some embodiments, the medical device is implantable. In some embodiments, the medical device can be disposed over an outer skin or epidermis of a patient, for example, can be applied to a wound for wound closure.

One aspect of the present disclosure relates to an alloy that has a transition metal or a salt thereof, the alloy being included in a medical device (e.g., in the inhibition layer 104 and/or the medical device body 102). Through galvanic corrosion or an ionic mechanism, the alloy can produce an effective amount of an ion or the salt of the transition metal to the environment surrounding the alloy. Another aspect of the present disclosure relates to a method of inhibiting the activity of a bacterial collagenase and/or a MMP, due to the degradation of an implanted medical device that has the alloy described herein. For example, as previously described herein, the transition metal or transition metal salt that may be included in the inhibition layer 104 and/or the medical device body 102 (e.g., encapsulated, embedded, or otherwise incorporated into the polymer and/or the medical device body 102) can include an alloy that generates ions or salt of the transition metal at an implantation or wound site (e.g., due to galvanic corrosion and/or ionic mechanism, or leaching out from the medical device 100). The generated ions inhibit bacterial collagenase and/or MMP.

In some embodiments, the alloy is configured to release an effective amount of an ion or the salt of the transition metal to a surrounding environment for inhibiting activity of a bacterial collagenase and/or a MMP under normal physiological conditions. In some embodiments, the local inhibition of the activity of bacterial collagenase, as well as the control of the MMPs due to transition metal ions or salts from the alloy can influence the rate of wound healing at implant or surgical sites. In some embodiments, the MMP can be MMP1, MMP8, or MMP9.

In some embodiments, the transition metal or a salt thereof can be embedded homogeneously in the alloy. The transition metal or a salt thereof can be embedded during the processing of the alloy which may include melt processing followed by vacuum homogenization or through powder metallurgy processes. In some embodiments the transition metal can be iron (Fe), manganese (Mn), silver (Ag), copper (Cu), or nickel (Ni). In some embodiments, the transition metal is not cobalt (Co). In some embodiments, the salt of the transition metal is FeCl₂, FeS, FeSe, MnCl₂, MnS, MnSe, CuCl₂, CuS, CuSe, NiCl₂, NiS, NiSe, or AgNO₃. In some embodiments, the salt of the transition metal is not CoCl₂.

In some embodiment, the alloy comprises at least 50% iron by weight, at least 25% manganese by weight, and at least 0.01% sulfur and/or selenium by weight, wherein the alloy is nonmagnetic. The sulfur or selenium can be dispersed homogeneously in the alloy.

In some embodiments, the alloy includes at least 55% iron by weight, e.g., at least 60% iron by weight, at least 65% iron by weight, or at least 70% iron by weight. In some embodiments, the alloy includes 50% to 75% iron by weight, e.g., 50% to 70% by weight, 50% to 60% iron by weight, 55% to 60% iron by weight, 55% to 70% iron by weight, or 60% to 70% iron by weight, inclusive. In some embodiments, the alloy includes 65% to 72% by weight of iron, inclusive.

In some embodiments, the alloy includes at least 28% manganese by weight, e.g., at least 30% manganese by weight, at least 35% manganese by weight, at least 40% manganese by weight, or at least 45% manganese by weight. In some embodiments, the alloy includes about 25% to about 45% manganese by weight, e.g., about 25% to about 40% manganese by weight, about 25% to about 35% manganese by weight, about 30% to about 45% manganese by weight, or about 35% to about 45% manganese by weight, inclusive of all ranges and values therebetween. In some embodiments, the alloy includes 28% to 35% by weight of manganese, inclusive.

In some embodiments, the alloy includes about 0.01% to about 2.0% sulfur by weight, inclusive. In some embodiments, the alloy includes about 0.01% to about 1.5% sulfur by weight, inclusive. In some embodiments, the alloy includes about 0.01% to about 1.2% sulfur by weight, inclusive. In some embodiments, the alloy includes about 0.01% to about 1.0% sulfur by weight, inclusive. In some embodiments, the alloy includes about 0.01% to about 0.35% sulfur by weight, inclusive. In some embodiments, the alloy includes about 0.01% to about 0.30% sulfur by weight, inclusive. In some embodiments, the alloy includes about 0.01% to about 0.20% sulfur by weight, inclusive. In some embodiments, the alloy includes about 0.01% to about 0.15% sulfur by weight, inclusive. In some embodiments, the alloy includes about 0.02% to about 0.10% sulfur by weight, inclusive. In some embodiments, the alloy includes about 0.10% to about 0.35% sulfur by weight, inclusive. In some embodiments, the alloy includes about 0.15% to about 0.35% sulfur by weight, inclusive. In some embodiments, the alloy includes about 0.20% to about 0.35% sulfur by weight, inclusive. In some embodiments, the alloy includes about 0.5% to about 2.0% sulfur by weight, inclusive. In some embodiments, the alloy includes about 0.5% to about 1.5% sulfur by weight, inclusive. In some embodiments, the alloy includes about 0.5% to about 1.2% sulfur by weight, inclusive. In some embodiments, the alloy includes about 0.5% to about 1.0% sulfur by weight, inclusive.

In some embodiments, the alloy includes about 0.01% to about 2.0% selenium by weight, inclusive. In some embodiments, the alloy includes about 0.01% to about 1.5% selenium by weight, inclusive. In some embodiments, the alloy includes about 0.01% to about 1.2% selenium by weight, inclusive. In some embodiments, the alloy includes about 0.01% to about 1.0% selenium by weight, inclusive. In some embodiments, the alloy includes about 0.01% to about 0.35% selenium by weight, inclusive. In some embodiments, the alloy includes about 0.01% to about 0.30% selenium by weight, inclusive. In some embodiments, the alloy includes about 0.01% to about 0.20% selenium by weight, inclusive. In some embodiments, the alloy includes about 0.01% to about 0.15% selenium by weight, inclusive. In some embodiments, the alloy includes about 0.02% to about 0.10% selenium by weight, inclusive. In some embodiments, the alloy includes about 0.10% to about 0.35% selenium by weight, inclusive. In some embodiments, the alloy includes about 0.15% to about 0.35% selenium by weight, inclusive. In some embodiments, the alloy includes about 0.20% to about 0.35% selenium by weight, inclusive. In some embodiments, the alloy includes about 0.5% to about 2.0% selenium by weight, inclusive. In some embodiments, the alloy includes about 0.5% to about 1.5% selenium by weight, inclusive. In some embodiments, the alloy includes about 0.5% to about 1.2% selenium by weight, inclusive. In some embodiments, the alloy includes about 0.5% to about 1.0% selenium by weight, inclusive.

In some embodiments, the alloy includes about 0.01% to about 2.0% sulfur and selenium by weight, inclusive. In some embodiments, the alloy includes about 0.01% to about 1.5% sulfur and selenium by weight, inclusive. In some embodiments, the alloy includes about 0.01% to about 1.2% sulfur and selenium by weight, inclusive. In some embodiments, the alloy includes about 0.01% to about 1.0% sulfur and selenium by weight, inclusive. In some embodiments, the alloy includes about 0.01% to about 0.35% sulfur and selenium by weight, inclusive. In some embodiments, the alloy includes about 0.01% to about 0.30% sulfur and selenium by weight, inclusive. In some embodiments, the alloy includes about 0.01% to about 0.20% sulfur and selenium by weight, inclusive. In some embodiments, the alloy includes about 0.01% to about 0.15% sulfur and selenium by weight, inclusive. In some embodiments, the alloy includes about 0.02% to about 0.10% sulfur and selenium by weight, inclusive. In some embodiments, the alloy includes about 0.10% to about 0.35% sulfur and selenium by weight, inclusive. In some embodiments, the alloy includes about 0.15% to about 0.35% sulfur and selenium by weight, inclusive. In some embodiments, the alloy includes about 0.20% to about 0.35% sulfur and selenium by weight, inclusive. In some embodiments, the alloy includes about 0.5% to about 2.0% sulfur and selenium by weight, inclusive. In some embodiments, the alloy includes about 0.5% to about 1.5% sulfur and selenium by weight, inclusive. In some embodiments, the alloy includes about 0.5% to about 1.2% sulfur and selenium by weight, inclusive. In some embodiments, the alloy includes about 0.5% to about 1.0% sulfur and selenium by weight, inclusive. The weight ratio of sulfur to selenium can be in the range of 99:1 to 1:99, inclusive. For example, the weight ratio of sulfur to selenium can be in the range of 99:1 to 75:1, 99:1 to 50:1, or 90:1 to 50:1, inclusive.

In some embodiments, the alloy includes at least 0.1% by weight niobium, e.g., about 0.125% by weight, 0.15% by weight, 0.175% by weight, 0.2% by weight, 0.225% by weight, 0.25% by weight, 0.275% by weight, or 0.3% by weight niobium. In some embodiments, the alloy includes at most 0.3% by weight niobium, e.g., at most 0.275% by weight, at most 0.25% by weight, at most 0.225% by weight, at most 0.2% by weight, at most 0.175% by weight, at most 0.15% by weight, or at most 0.125% by weight niobium. In some embodiments, the alloy includes in a range of 0.1% to 0.3% by weight niobium, e.g., 0.15% to 0.25% by weight niobium, or 0.175% to 0.225% by weight niobium. The niobium may serve as a grain size stabilizer to cause a more uniform grain structure in the alloy.

In some embodiments, the alloy includes about 50% to about 70% iron by weight, about 25% to about 35% manganese by weight, and about 0.01% to about 0.35% sulfur by weight.

In some embodiments, the alloy includes about 50% to about 70% iron by weight, inclusive, about 25% to about 35% manganese by weight, inclusive, and about 0.01% to about 0.35% selenium by weight, inclusive.

In some embodiments, the alloy includes about 50% to about 70% iron by weight, inclusive, about 25% to about 35% manganese by weight, inclusive, and about 0.01% to about 0.35% sulfur and selenium by weight, inclusive. The weight ratio of sulfur to selenium can be in the range of 1:99 to 99:1, inclusive. For example, the weight ratio of sulfur to selenium can be in the range of 99:1 to 75:1, 99:1 to 50:1, or 90:1 to 50:1, inclusive.

In some embodiments, the alloy includes Mn in a range of 28% to 35% by weight, inclusive, Fe in a range of 65% to 72% by weight, inclusive, and Nb in a range of 0.1% to 0.3% by weight, inclusive. In some embodiments, any of the alloys described herein may include carbon in a range of 0.06% to 0.1% by weight, inclusive (e.g., 0.06%, 0.07%, 0.08%, 0.09%, or 0.1% by weight of carbon, inclusive).

In some embodiments, the concentration of sulfur and/or selenium in the alloy can be adjusted to control the degradation rate of the alloy. The higher the concentration of sulfur and/or selenium, the faster the degradation rate. In some embodiments, the alloy may include sulfur and/or selenium in a range of about 100 parts per million (ppm) to 6,000 parts ppm, inclusive. For example, the alloy may include sulfur and/or selenium in a range of about 300 ppm to about 3,000 ppm, inclusive.

In some embodiments, the alloy may or may not contain minor additions of carbon, nitrogen, phosphorous, silicon, or trace elements typically associated with Fe—Mn alloys. In some embodiments, the alloy is substantially free of chromium. In some embodiments, the alloy is substantially free of nickel.

In some embodiments, the alloy may include any of the alloys described in U.S. application Ser. No. 16/095,104, filed Oct. 5, 2018, and entitled “Fe—Mn Implantable Alloys with Enhanced Degradation Rates, the entire disclosure of which is incorporated herein by reference.

The alloy can be in any shape or form needed in conformity with the function of the medical device. In some embodiments, the alloy can be partially or fully embedded in or interwoven with a polymer as described herein to form the medical device. For example, the alloy can be in the form of nanoparticles or microparticles, which are partially or fully embedded in a polymer to form the medical device. In some embodiments, the alloy can be in the form of a coating.

In some embodiments, the medical device (e.g., the medical device 100) comprising the alloy may be in the form of a bone screw, a bone anchor, a tissue staple, a craniomaxillofacial reconstruction plate, a surgical alloy mesh, a composite surgical mesh consisting of woven alloy and natural or synthetic fibers, a surgical closure, a bioabsorbable suture, a non-bioabsorbable suture, a fastener, a reconstructive dental device, a component of a tissue sealant/glue, or a stent.

In some embodiments, the medical device (e.g., the medical device 100) comprising the alloy is implantable. In some embodiments, the medical device can be applied to a wound for wound closure. In some embodiments, the medical device comprising the alloy is implanted in or applied to a subject in need thereof to inhibit the activity of a bacterial collagenase and/or a MMP. In some embodiments, the alloy experiences corrosion (e.g., galvanic corrosion, or leaching), resulting in the local liberation of transition metal ions or salts in the local tissue fluid when used as tissue fixation devices, or in the local area of where the medical device is implanted or otherwise disposed.

In some embodiments, the medical device (e.g., the medical device 100) comprising the polymer and/or alloy has the requisite tensile strength, elongation, and ductility to perform in similar fashion to the currently used titanium tissue fixation devices (e.g., staples, clips, sutures, etc.). For example, a mesh fabricated from the alloy can have a yield strength between 55,000 and 65,000 psi and a tensile strength of around 90,000 psi (+/−5,000 psi). In some embodiments, a stent fabricated from the alloy may have a yield strength between 50,000 psi and 90,000 psi, and a tensile strength in a range of 80,000 psi to about 150,000 psi, inclusive. The medical device can be suitable for implantation in the gastrointestinal tract, soft tissue, or bone of a subject.

In some embodiments, the effective amount of an ion or a salt of the transition metal released by the medical device (e.g., the medical device body 102 and/or the inhibition layer 104) is in a range of about 10 micromolar to about 120 micromolar, inclusive. In some embodiments, the effective amount of the ion or the salt of the transition metal is in a range of about 20 micromolar to about 100 micromolar, inclusive (e.g., about 10 micromolar, about 20 micromolar, about 30 micromolar, about 40 micromolar, about 50 micromolar, about 60 micromolar, about 70 micromolar, about 80 micromolar, about 90 micromolar, about 100 micromolar, about 110, or about 120 micromolar, inclusive of all ranges and values therebetween). In some embodiments, the effective amount of the ion or the salt of the transition metal is at least about 20 micromolar. In some embodiments, the effective amount of the ion or the salt of the transition metal is at least about 30 micromolar. In some embodiments, the effective amount of the ion or the salt of the transition metal is at least about 40 micromolar. In some embodiments, the effective amount of the ion or the salt of the transition metal is at least about 50 micromolar. In some embodiments, the effective amount of the ion or the salt of the transition metal is at least about 60 micromolar. In some embodiments, the effective amount of the ion or the salt of the transition metal is at least about 70 micromolar. In some embodiments, the effective amount of the ion or the salt of the transition metal is at least about 80 micromolar. In some embodiments, the effective amount of the ion or the salt of the transition metal is at least about 90 micromolar.

In some embodiments, the effective amount of the ion or the salt of the transition metal is at most about 120 micromolar. In some embodiments, the effective amount of the ion or the salt of the transition metal is at most about 100 micromolar. In some embodiments, the effective amount of the ion or the salt of the transition metal is at most about 90 micromolar. In some embodiments, the effective amount of the ion or the salt of the transition metal is at most about 80 micromolar. In some embodiments, the effective amount of the ion or the salt of the transition metal is at most about 70 micromolar. In some embodiments, the effective amount of the ion or the salt of the transition metal is at most about 60 micromolar. In some embodiments, the effective amount of the ion or the salt of the transition metal is at most about 50 micromolar. In some embodiments, the effective amount of the ion or the salt of the transition metal is at most about 40 micromolar.

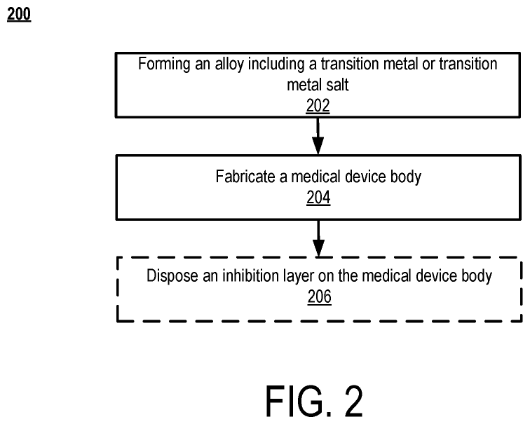

FIG. 2 is a schematic flow chart of a method 200 for manufacturing a medical device such as an implantable medical device (e.g., the medical device 100), according to an embodiment. While described with respect to the medical device 100, the method 200 may be used to form any medical device including the transition metal and/or transition metals salts described herein.

The method 200 includes forming an alloy including a transition metal, or transition metal alloy (e.g., Mn, Fe, and/or any of the transition metals or transition metal salt, as described herein), at 202. At 204, a medical device body (e.g. the medical device body 102) is fabricated. In some embodiments the medical device body 102 may include or incorporate the alloy, or may be formed from the alloy.

For example, an ingot of a metal or metal alloy is melted. The metal or metal alloy may include an ingot of a transition metal or include a transition metal or a transition metal salt. In some embodiments, the alloy may include in a range of 28% to 35% by weight Mn, in a range of 65% to 72% by weight Fe, 0.1% to 0.3% Nb, and 0.06% to 0.1% by weigh C, as previously described herein. In some embodiments, the alloy may include any of the alloy including any concentration of a transition metal and/or transition metal salt, as previously described herein.

The metal or metal alloy may be melted by induction melting under vacuum or substantial vacuum, and in a furnace that may be backfilled with an inert gas (e.g., about 100 mm-Hg to about 300 mm-Hg of Argon, inclusive). For example, the furnace may initially be maintained at vacuum and once the ingot starts to melt, the furnace may be backfilled with the inert gas. The melted metal or metal alloy may be poured into molds (e.g., ingot molds) and cooled. In some embodiments, the mold may have a diameter in a range of about 2 inches to about 4 inches, inclusive (e.g., 2.0, 2.5, 3.0, 3.5, 4.0, 4.5, or 5.0 inches, inclusive). The melted metal or metal alloy may be cooled under an inert gas at any suitable back pressure (e.g., under Argon gas at a pressure in a range of 600 mm-Hg to about 1,000 mm-Hg, inclusive).

In some embodiments, the molded metal or metal alloy may be homogenized. For example, the molded metal or metal alloy may bae homogenized at a temperature in a range of about 1,000 degrees Celsius to about 1,200 degrees Celsius, inclusive (e.g., 1,000, 1,025, 1,050, 1,075, 1,100, 1,125, 1,150, 1,175, or 1,200 degrees Celsius, inclusive) under vacuum, for a time in a range of about 10 hours to 12 hours, inclusive. The homogenization may allow component migration (i.e., molecular migration of the elements included in the alloy) that may increase uniformity, thus yielding a more uniform solid solution of alloying elements. In some embodiments, the homogenized metal or metal alloy may be hot worked. For example, the homogenized metal or metal alloy may be hot worked by extruding at a temperature in a range of about 800 degrees Celsius to about 1,000 degrees Celsius, inclusive (e.g., about 800, 825, 850, 875, 900, 925, 950, 975, or 1,000 degrees Celsius, inclusive) to reduce the diameter thereof from a first diameter (e.g., about 3.5 inches) to a second diameter (e.g., about 1 inch).

In some embodiments, the hot worked metal or metal alloy may be cold drawn and annealed. For example, the hot worked metal or metal alloy may be serially cold drawn (i.e., subjected to one or more sequential cold drawing and annealing operations) to a third diameter (e.g., less than 1 inches such as about 0.25 inches) so as to break down the grain structure and obtain a desired grain size and to further increase uniformity of alloying content distribution in the metal or metal alloy. In some embodiments, the grain size of may be less 10 microns, less than 5 microns, or less than 2 microns. In some embodiments, the grain size may be in a range of about 1 micron to about 10 microns, inclusive. In some embodiments, the grain size may be in a range of about 1 micron to about 8 microns, inclusive. In some embodiments, the grain size may be in a range of about 1 micron to about 5 microns, inclusive. In some embodiments, the grain size may be in a range of about 1 micron to about 3 microns, inclusive. In some embodiments, the grain size may be in a range of about 1 microns to about 2 microns, inclusive. In some embodiments, the grain size may by in a range of about 2 microns to about 8 microns, inclusive. In some embodiments, the grain size may by in a range of about 2 microns to about 5 microns, inclusive. The annealed metal or metal alloy may be ground (e.g., centerless ground) to the final size (e.g., a desired size of the medical device body 102) and may then be further annealed to yield a desired strength (e.g., a yield strength in a range of about 50,000 psi to 90,0000 psi, and an ultimate tensile strength in a range of about 80,000 psi to about 150,000 psi). In some embodiments, the homogenized ingot may be remelted (e.g., electroslag remelted) to remove contaminants and/or add alloying components such as transition metals or transition metal salts (e.g., Mn, Ag, Cu, Ni, S, Se, FeCl₂, FeS, FeSe, MnCl₂, MnS, MnSe, CuCl₂, CuS, CuSe, NiCl₂, NiS, NiSe, any other suitable alloying component or a combination thereof).

In some embodiments, fabricating the medical device body 102 may include molding, homogenizing, hot working, cold drawing, annealing, remelting, and/or ground the metal or metal alloy to form the medical device body 102. In some embodiments, medical device body may be formed by forming a wire of the alloy in operation 202, and shaping the wire into a desired shape of the medical device body. In some embodiments, the method 200 may include disposing the inhibition layer 104 on the medical device body 102. For example, the formed metal alloy including the transition metal and/or transition metal salt may be coated on the medical device body 102, for example, powder deposited, or broken down into small particles that are embedded or incorporated into a polymer that is disposed on the medical device body 102, as previously described. In this manner, the medical device 100 is formed that includes the transition metal within the medical device body 102 and/or the inhibition layer 104 disposed thereon so as to inhibit bacterial collagenase and/or MMP. The medical device 100 may be implanted in the body of the patient or disposed on a tissue of the patient (e.g., used as a staple to close a wound). The transition metal and/or transition metal salts included in the medical device 100 leech or migrate from the medical device body 102 (e.g., due to corrosion, ion migration, etc.) to inhibit bacterial collagenase and/or MMP, thereby reducing healing time and reducing infection risks.

### EXPERIMENTAL EXAMPLES

Following are examples of alloys configured to inhibit bacterial collagenase and/or MMPs according to the embodiments described herein. These examples are for illustrative purposes only and should not be construed as limiting the scope of the disclosure.

### Example 1

An experiment was performed to compare the effects of Fe—Mn alloy on the effect of bacterial collagenase growth vs a control solution without alloy in an aqueous solution using a standard assessment of E. faecalis derived collagenase activity. This model replicates the early surface corrosion effect which occurs immediately upon exposure of the alloys surface to an aqueous solution as occurs in clinical tissue fixation. Wire samples were prepared by cutting about 0.25 mm diameter titanium and iron-manganese wire to about 2 cm lengths. Three wire pieces from each alloy were placed in separate culture tubes with tryptic soy broth and inoculated with E. faecalis to make 2×10⁷ colony forming units (CFU). The samples were incubated at 37° C. with shaking for 24 hours. After incubation, the samples were centrifuged to remove the bacterial culture and analyzed for collagenase activity using the EnzChek Gelatinase/Collagenase Assay Kit. The two samples incubated with the iron-manganese alloy showed highly significant inhibition of collagenase activity compared to the control solution which demonstrated extensive collagenase activity. Microbiologic studies were also performed using wire sections against standard cultures of several collagenase secreting bacteria, using a zone of inhibition analysis. There was no evidence of antimicrobial activity exhibited by the Fe—Mn alloy material.

### Example 2

An experiment was performed using wire made from an alloy of 28 weight percent manganese (Mn) and the balance iron (Fe), to measure the contribution of Fe and Mn from a wire length to solution volume ratio of about 2 cm of about 0.25 mm wire to about 2 ml of solution (e.g., 1 cm length per ml), of the wire utilized in the E. Faecalis derived collagenase study of Example 1-1. The wire was incubated in phosphate buffered saline, at about 37° C. for about 24 hours and analyzed by inductively coupled plasma-mass spectrometry (ICP-MS) for Fe and Mn concentrations. The solution was determined to contain about 33.53 micromolar iron and about 35.00 micromolar manganese. The experiment demonstrated that 33.53 μM Fe and 35.00 μM Mn are sufficient to prevent the activation of E. faecalis derived collagenase.

### Example 3

A significant passivation layer forms on the alloy when placed in tissue, which effectively retards corrosion to avoid any significant reduction in implant tensile strength allowing support of tissue healing to proceed safely. Prior animal studies, using a colorectal anastomosis porcine model demonstrated early and complete mucosal healing coupled with excellent healing of all bowel wall layers. Serum analysis showed no evidence of systemic rises in either iron or manganese. Taken together these data strongly support an effective early local liberation of sufficient iron and manganese ions, or other similar ions to interfere with local activity of bacterial collagenase and by extension MMP function. The formation of an effective passivation layer of organic molecules on the device and demonstrated early mucosal healing potentially limits the duration of ionic activity to the time of exposure of the unhealed wound to luminal bacteria and the related enzyme activity. This is the only period of time during which bacterial collagenase can up-regulate the activity of MMP's.

### Example 4

An experiment was performed with various lengths of about 0.25 mm diameter iron-28% manganese wire alloy in a ratio of wire length to solution volume equivalent to the ratio of wire length to tissue volume of an anastomotic staple line, specifically, 19.2 cm wire length of 0.25 mm diameter wire per 10 cc of tissue. Wire samples were incubated at 37° C. for 1 day, 3 days and 7 days. The resulting solutions were analyzed by ICP-MS for Fe and Mn concentration. Table 1 depicts micromolar concentrations of iron and manganese in solution.

TABLE 1
Tissue equivalent micromolar concentrations (μM)

| Incubation Time | One Day | Three Days | Seven Days |
|---|---|---|---|
| Iron (Fe) | 30.47 μM | 68.86 μM | 63.40 μM |
| Manganese (Mn) | 38.61 μM | 52.87 μM | 58.32 μM |

One day of incubation at a ratio equivalent to the tissue surrounding a surgical staple line, consisting of the tissue trapped by the staple plus an equivalent radius surrounding the staple line, produced a concentration of 30.47 micromolar Fe and 38.61 micromolar manganese, which increased to 68.86 micromolar Fe and 52.87 micromolar Mn at three days and remained relatively constant through seven days. The presence of the Fe and Mn in the tissue is indicative of the potential of using this alloy for inhibiting bacterial collagenase and/or MMP.

### Example 5

An experiment laparotomy was performed on four pigs to assess the healing of a surgical rectal closure using staples made from a test alloy that included 28% manganese by weight, 0.08% carbon by weight, 0.2% niobium by weight, and the balance being iron. Under general anesthesia, an end-to-end circular stapler, loaded with staples fabricated from the test alloy, was inserted into the rectum and an anastomosis created with the descending colon. The anastomoses were tested for leaks using standard insufflation testing with contrast media under x-ray visualization—with no leaks detected. Leak testing was repeated at 14 and 30 days on one pig with no leaks detected. Leak testing was repeated on all animals at 60 day follow up, with no anastomotic leaks detected. There were no adverse events during the 60 day follow up period, test animals remained healthy with no evidence of anastomotic leaks, fever or other signs of illness. All animals were harvested 60 days post-surgery. The treated tissue was resected for both gross and histologic examination. Anastomotic tissue demonstrated improved healing as evidenced by a virtually seamless anastomosis without an anastomotic lip and no adverse histologic tissue conditions.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

Definitions, as defined and used herein, should be understood to control over dictionary definitions, definitions in documents incorporated by reference, and/or ordinary meanings of the defined terms.

The term “comprising” as used herein is synonymous with “including” or “containing” and is inclusive or open-ended and does not exclude additional, unrecited members, elements or method steps. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The articles “a” and “an” are used in this disclosure to refer to one or more than one (i.e., to at least one) of the grammatical object of the article. By way of example, “an element” means one element or more than one element. The term “and/or” is used in this disclosure to mean either “and” or “or” unless indicated otherwise.

The phrase “and/or,” as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein, “or” should be understood to have the same meaning as “and/or” as defined above. For example, when separating items in a list, “or” or “and/or” shall be interpreted as being inclusive, i.e., the inclusion of greater than one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, the term “about” means within ±10% of a given value or range.

As used herein, the terms “biodegradable,” “bioabsorbable,” and “bioresorbable” all refer to a material that is able to be chemically broken down in a physiological environment, i.e., within the body or inside body tissue, such as by biological processes including resorption and absorption. This process of chemical breakdown will generally result in the complete degradation of the material within a period of weeks to months, such as 18 months or less, 24 months or less, or 36 months or less, for example. This rate stands in contrast to more “degradation-resistant” or permanent materials, such as those constructed from nickel-titanium alloys (“Ni—Ti”) or stainless steel, which remain in the body, structurally intact, for a period exceeding at least 36 months and potentially throughout the lifespan of the recipient.

As used herein, the term “substantially free” when referring to the presence of an element in an alloy means that the concentration of the element in the alloy is no more than 0.1%, no more than 0.05% by weight, or no more than 0.01% by weight.

As used herein, the term “inhibit,” “inhibition,” or “inhibiting” refers to the reduction or suppression of a biological activity (e.g., an enzymatic activity). The reduction of the biological activity can be at least about 10%, at least about 20%, at least about 30%, at least about 40%, at least about 50%, at least about 60%, at least about 70%, at least about 80%, or at least about 90%.

1. A medical device, comprising:
a polymer including at least one of a transition metal or a transition metal salt,
wherein under normal physiological conditions, the polymer is configured to release an effective amount of an ion or the salt of the transition metal to a surrounding environment for inhibiting activity of a bacterial collagenase and/or a matrix metalloproteinase (MMP).

2. The medical device of claim 1, comprising:
a medical device body; and
an inhibition layer disposed on the medical device body, the inhibition layer comprising the polymer.

3. The medical device of claim 1, wherein the at least one of the transition metal or the transition metal salt is embedded homogeneously in the polymer.

4. The medical device of claim 1, wherein the transition metal is in the form of an alloy.

5. The medical device of claim 1, wherein the transition metal is iron (Fe), manganese (Mn), silver (Ag), copper (Cu), or nickel (Ni).

6. The medical device of claim 1, wherein the transition metal salt is FeCl₂, FeS, FeSe, MnCl₂, MnS, MnSe, CuCl₂, CuS, CuSe, NiCl₂, NiS, or NiSe.

7. The medical device of claim 1, wherein the transition metal salt is not CoCl₂.

8. The medical device of claim 1, wherein the MMP is MMP1, MMP8, or MMP9.

9. The medical device of claim 1, wherein the medical device is in the form of a bone screw, a bone anchor, a tissue staple, a craniomaxillofacial reconstruction plate, a surgical alloy mesh, a composite surgical mesh consisting of woven alloy and natural or synthetic fibers, a surgical closure, a fastener, a reconstructive dental device, a component of a tissue sealant/glue, or a stent.

10. The medical device of claim 1, wherein the medical device is implantable.

11. A method of inhibiting activity of a bacterial collagenase and/or a matrix metalloproteinase (MMP) in a subject in need thereof, the method comprising applying to or implanting in the subject, a medical device that has an alloy, wherein:
the alloy includes at least one of a transition metal or a transition metal salt and
under normal physiological conditions, the alloy is configured to release an effective amount of an ion or the salt of the transition metal to a surrounding environment for inhibiting activity of the bacterial collagenase and/or the MMP.

12. The method of claim 11, wherein the at least one of the transition metal or the transition metal salt is embedded homogeneously in the alloy.

13. The method of claim 11, wherein the transition metal is iron (Fe), manganese (Mn), silver (Ag), zinc (Zn), copper (Cu), or nickel (Ni).

14. The method of claim 11, wherein the transition metal salt is FeCl₂, FeS, FeSe, MnCl₂, MnS, MnSe, CuCl₂, CuS, CuSe, NiCl₂, NiS, or NiSe.

15. The method of claim 11, wherein the transition metal salt is not CoCl₂.

16. The method of claim 11, wherein the MMP is MMP1, MMP8, or MMP9.

17. The method of claim 11, wherein the medical device is in the form of a bone screw, a bone anchor, a tissue staple, a craniomaxillofacial reconstruction plate, a surgical alloy mesh, a composite surgical mesh consisting of woven alloy and natural or synthetic fibers, a surgical closure, a fastener, a reconstructive dental device, a component of a tissue sealant/glue, or a stent.

18. The method of claim 11, wherein the alloy comprises at least 50% iron by weight, at least 25% manganese by weight, and at least one of 0.01% sulfur or selenium by weight, and wherein the alloy is nonmagnetic.

19. The method of claim 18, wherein the alloy comprises at least 60% Fe by weight.

20. The method of claim 18, wherein the alloy comprises at least 30% Mn by weight.

21. The method of claim 18, wherein the alloy comprises Mn in a range of 28% to 35% by weight, Fe in a range of 65% to 72% by weight, and Nb in a range of 0.1% to 0.3% by weight.

22. The method of claim 11, wherein the medical device is implanted in the gastrointestinal tract, soft tissue, or bone of the subject.

23. The method of claim 11, wherein the effective amount is in a range of about 10 micromolar to about 120 micromolar.

24. A medical device, comprising:
a medical device body formed from a transition metal alloy including Mn in a range of 28% to 35% by weight, Fe in a range of 65% to 72% by weight, and Nb in a range of 0.1% to 0.3% by weight, the medical device configured to be implanted in the body of a patient or applied to a tissue of the patient such that under normal physiological conditions, the medical device body is configured to release an effective amount of the Mn and Fe in the surrounding tissue for inhibiting activity of a bacterial collagenase and/or a matrix metalloproteinase (MMP).

25. The medical device of claim 24, wherein the effective amount of Fe and Mn in the surrounding tissue is in a range of about 10 μM to about 120 μM.

26. The medical device of claim 25, wherein the effective amount of Fe and Mn in the surrounding tissue is in a range of about 40 μM to about 80 μM after at least 3 days of the medical device being in contact with the surrounding tissue.

27. The medical device of claim 24, wherein the medical device is in the form of a bone screw, a bone anchor, a tissue staple, a craniomaxillofacial reconstruction plate, a surgical alloy mesh, a composite surgical mesh consisting of woven alloy and natural or synthetic fibers, a surgical closure, a fastener, a reconstructive dental device, a component of a tissue sealant/glue, or a stent.

28. The medical device of claim 24, wherein the medical device has a yield strength in a range of about 50,000 psi to about 90,000 psi.

29. The medical device of claim 28, wherein the ultimate yield strength of the medical device is in a range of about 80,000 psi to about 150,000 psi.
